(19) United States
(12) Patent Application Publication
(10) Pub. No.: US 2025/0354187 A1
(43) Pub. Date: Nov. 20, 2025

(54) High-Yield Method and Kit for Preparing mRNA by Reducing or Inhibiting Double-Stranded RNA Formation During In Vitro Transcription

(71) Applicant: Institute of Process Engineering, Chinese Academy of Sciences, Beijing (CN)

(72) Inventors: Songping Zhang, Beijing (CN); Zhiguo Su, Beijing (CN); Xue Feng, Beijing (CN); Zhengjun Li, Beijing (CN); Yanyan Ma, Beijing (CN)

(21) Appl. No.: 19/170,081

(22) Filed: Apr. 4, 2025

Related U.S. Application Data
(63) Continuation of application No. PCT/CN2024/091989, filed on May 9, 2024.

(30) Foreign Application Priority Data
May 11, 2023 (CN) 202310528321.8
May 11, 2023 (CN) 202310531556.2

(51) Int. Cl. C12P 19/34

(52) U.S. Cl. CPC C12P 19/34

(57) ABSTRACT

Provided is a high-yield method and kit for preparing mRNA by reducing or inhibiting double-stranded ribonucleic acid (dsRNA) formation during in vitro transcription. The preparation method is to add solid phase media during a transcription process. Compared with the existing technology, the present invention has the following advantages: according to the high-yield method and kit for preparing mRNA, different types of negatively charged solid phase media are added during the in vitro transcription, reducing the production of dsRNA by interface regulation, and improving the yield and stability of mRNA; in addition, the transfection efficiency of the mRNA prepared by solid phase regulation is improved, and the expression of immune factors is reduced. The solid phase media used in the method and kit are insoluble in water and do not contaminate the transcription system; after the transcription is completed, the solid phase media can be easily separated, and the operation is simple; after proper treatment, the solid phase media can be reused, thus the method and kit have low costs and can be easily scaled up to industrial-scale production.

### FIELD

The present invention relates to the technical fields of in vitro transcription synthesis of mRNA and separation and purification of ribonucleic acid. Specifically, it involves a high-yield preparation method and a kit for mRNA that can reduce or inhibit the formation of double-stranded ribonucleic acid during the in vitro transcription process.

### BACKGROUND

mRNA vaccines are new technologies that combine molecular biology and immunology and are closely related to gene therapy. In the past decade, mRNA vaccines have been effectively used for immunization against influenza viruses, Zika viruses and rabies viruses. Especially after the outbreak of the COVID-19 pandemic, mRNA vaccines have gradually become a research hotspot due to their advantages such as rapid research and development speed, high safety, scalability, and high efficiency. Non-replicating mRNA is usually prepared by in vitro transcription. Using linearized plasmid DNA as a template, the target mRNA is synthesized through an enzymatic reaction by the action of RNA polymerase. Then, capping is carried out at the 5′ end and polyadenylation is performed at the 3′ end of the mRNA. Therefore, the samples obtained through in-vitro transcription usually contain impurities such as RNA polymerase, residual NTPs, DNA templates, dsRNA and abnormally terminated mRNA. Among them, the dsRNA impurities have a great impact on the efficacy and safety of mRNA vaccines, such as reducing translation efficiency, causing inflammatory responses and immune stress responses. Therefore, it is particularly important to reduce dsRNA in mRNA products.

During in-vitro transcription, the formation of dsRNA is mainly based on two mechanisms. The first is based on RNA-dependent RNA polymerase. For the mRNA produced by in-vitro transcription, if there is a certain complementarity at 3′-end, it may fold back. Under the action of T7 polymerase, it extends with the target RNA as a template to form a cis-3′-end-extended dsRNA. In addition, short transcripts specifically bind to the complementary sequences of the target mRNA under annealing conditions to form short-transcript-dsRNA. The second is based on DNA-dependent RNA polymerase that is independent of the promoter. Transcription takes the non-template strand as a template. Under the action of the T7 polymerase that is independent of the promoter, the antisense strand RNA is transcribed and forms dsRNA by complementing with the target RNA. Currently, the methods for removing dsRNA from the transcription products include purifying the mRNA to remove dsRNA after in-vitro transcription or reducing the production of dsRNA during the in-vitro transcription process. Markus et al. made dsRNA specifically bind to cellulose in the ethanol system through the method of cellulose chromatography, reducing the level of dsRNA by more than 90%. US20200071689A1 discloses a method for removing dsRNA from in vitro transcription products. RNase III is added to the transcribed mRNA system to digest the dsRNA in the products. This method can protect the mRNA from being digested by the enzyme while removing the dsRNA. However, in this method, the purification or enzymatic hydrolysis process may accidentally damage the secondary structure of the mRNA itself, reducing the integrity of the mRNA. Therefore, it is still necessary to remove this enzyme after the process, which increases the process cost, reduces the yield, and has relatively high limitations. Katalin et al. separated dsRNA from mRNA by HPLC method, and the translation level of mRNA in cells was increased by 10 to 1000 times. Although these purification methods can reduce the level of dsRNA in mRNA, mRNA is unstable and prone to degradation during the purification process.

Reducing the production of dsRNA during in-vitro transcription is to reduce the level of dsRNA from the source. At present, the main research is carried out in the following three aspects. (1) DNA template sequence reconstruction and modification. Adding a polyA-tail sequence to the DNA template can reduce the production of antisense-type dsRNA. During RNA synthesis, N1-methyl-pseudouridine-modified RNA may help to reduce the synthesis of antisense RNA chains, increase protein expression and reduce immunogenicity. By adding a DNA sequence complementary to 3′-end and competitively capturing DNA, self-extension at 3′-end can be effectively prevented and the production of dsRNA with self-extension at 3′-end can be reduced. (2) Reconstruction and modification of RNA polymerase. MONICA et al. used heat-resistant RNAP to carry out in-vitro transcription reactions at high temperatures, and 3′-end-extended dsRNA was significantly reduced, and the mRNA products showed lower immunogenicity. Heng Xia et al. found that the level of dsRNA in RNA synthesized by RNA polymerase encoded by psychrophilic bacteriophage VSW-3 was significantly reduced at low temperatures (4-25° C.), and 3′-end-extended and full-length dsRNA were almost completely eliminated. Moderna modified the amino acid sequence of T7 polymerase, and after the G47A+884G mutation of T7 polymerase, the production of dsRNA could be reduced. (3) Regulate the in vitro transcription process. Reducing the magnesium ion concentration to below 5 mM during in-vitro transcription can reduce the production of 3′-end-extended and antisense dsRNA. Conduct in-vitro transcription reactions under high-salt conditions to reduce the production of dsRNA by inhibiting the rebinding of RNA products, and immobilize the promoter DNA and T7 RNA polymerase to increase the total yield and purity of the encoded RNA. CN115087456 discloses a method for reducing the formation of double-stranded RNA in a transcription system. Adding at least one chaotropic agent to the transcription initiation reaction mixture can reduce or inhibit the interaction between bases and reduce the formation of dsRNA during RNA preparation. These methods can all reduce the production of dsRNA to a certain extent. However, high-temperature transcription and specific competition sites will increase the economic burden on mRNA production, especially in industrial production. Although chaotropic salts can reduce the synthesis of dsRNA, as a commonly-used protein-denaturing agent, they will affect the structure and activity of the T7 enzyme. In addition, the addition of a denaturing agent will also introduce new impurities. Besides, reducing the magnesium ion concentration in the transcription system and adding a denaturing agent will also affect the yield of mRNA. Therefore, it is particularly important to find other methods that can reduce the production of dsRNA without affecting in-vitro transcription and the T7 enzyme.

In conclusion, at present, the methods used to remove and reduce the level of dsRNA in the transcription system have problems such as complex operation, high cost, low mRNA yield and instability. How to provide a method that can efficiently reduce dsRNA, achieve a high mRNA yield, have a low cost and ensure good mRNA stability, and then turn it into a product (that is, solidify it into a kit), has become one of the urgent problems to be solved in the field of mRNA in vitro synthesis and separation and purification technology. The present invention proposes a method of adding a negatively charged solid-phase medium during the in vitro transcription process to reduce the yield of dsRNA and improve the yield and stability of mRNA. The solid-phase medium used in this method is insoluble in water and will not contaminate the transcription system; after the transcription is completed, the solid-phase medium is easy to separate, and the operation is simple; after appropriate treatment, the solid-phase medium can be reused, with low cost and is easy to be used in large-scale production. It is also surprisingly found that the transfection efficiency of mRNA prepared by solid-phase control is increased, and the expression of immune factors is somewhat reduced.

### SUMMARY

In view of the above-mentioned technical limitations, namely the problems of complex operation, high cost, low mRNA yield and poor stability in the current methods for removing dsRNA from mRNA, the present invention proposes a high-yield preparation method for mRNA that reduces or inhibits the formation of double-stranded ribonucleic acid (dsRNA) during in vitro transcription. It realizes simple and efficient reduction of dsRNA production, improves the yield of mRNA, and ensures the stability of mRNA. This method has the technical advantages of simple operation, no pollution, low cost, easy scale-up application, and the solid-phase medium can be reused. At the same time, the present invention also develops a kit for in vitro transcription synthesis of mRNA that can reduce or inhibit the formation of dsRNA based on the above method, overcoming the deficiencies and defects mentioned in the background technology.

To achieve the above objectives, the following technical solutions are adopted in this application:

The inventive point of this application is to provide a high-yield preparation method for mRNA that reduces or inhibits the formation of double-stranded ribonucleic acid during in vitro transcription. The preparation method involves adding a solid-phase medium during the transcription process.

Optionally, in the above high-yield mRNA preparation method, the solid-phase medium is a medium modified with negatively charged groups.

This modification can be at any position of the solid-phase medium. For example, negatively charged groups can be modified on the surface of the solid-phase medium, or negatively charged groups can be modified inside the solid-phase medium, etc.

Optionally, in the above high-yield mRNA preparation method, the negatively charged groups modified on the solid-phase medium are one or more of sulfonic acid group —SO₃, methylsulfonic acid group —CH₂SO₃, ethylsulfonic acid group —(CH₂)₂SO₃, propylsulfonic acid group —(CH₂)₃SO₃, phosphate group PO₃, carboxylic acid group —COO, formyl group —CH₂COO, hydroxyl group —OH, polyadenylic acid Ploy A, polythymidylic acid Ploy T, polyuridylic acid Ploy U, polyguanylic acid Ploy G, and polycytidylic acid Ploy C.

Wherein, the degree of polymerization (n) of Ploy A (polyadenylic acid), Ploy T (polythymidylic acid), Ploy U (polyuridylic acid), Ploy G (polyguanylic acid), and Ploy C (polycytidylic acid) is 1 to 100; and the degrees of polymerization and selections are 1, 5, 10, 15, 20, 25, 50, 75, and 100.

Optionally, in the above high-yield mRNA preparation method, the form of the solid-phase medium is one or more of granular, membranous and sheet-like.

Optionally, in the above high-yield mRNA preparation method,

The granular solid-phase medium includes one or more of microspheres and nanoparticles;

The material of the granular solid-phase medium includes one or more of organic materials, inorganic materials and functional materials;

The organic material includes one or more of natural polysaccharides and synthetic polymers;

The natural polysaccharide organic materials include one or more of cellulose, dextran, agarose, chitosan and konjac glucomannan;

The synthetic polymers include one or more of styrenic polymers, acrylic polymers and polyvinyl acid-type polymers;

The inorganic materials include one or more of silica gel, glass, metal oxides and hydroxyapatite;

The functional materials include one or more of magnetic materials and thermos-sensitive materials;

The membranous solid-phase medium includes one or more of nitrocellulose membrane, nylon membrane and glass-cellulose membrane;

The sheet-like solid-phase medium includes carbon nanotubes.

Optionally, the above-mentioned method for high-yield preparation of mRNA includes the following steps:
S1. Pretreatment of Solid-Phase Medium: Wash the solid-phase medium with sterile and nuclease-free water, then equilibrate it with a buffer solution. After shaking for equilibration, drain it by suction. Preferably, the buffer solution includes one or more of Tris-HCl buffer, citrate buffer, acetate buffer, phosphate buffer and HEPES buffer. The shaking equilibration time is 5-240 minutes. The equilibrated solid-phase medium is drained by suction through a sintered glass filter. The buffer solution is preferably Tris-HCl buffer or phosphate buffer. The concentration of the buffer solution is 0.01-1 M, and can be selected as 0.01 M, 0.05 M, 0.1 M, 0.3 M, 0.5 M, 0.7 M, 1 M. The shaking equilibration time is preferably 60-240 minutes, and can be selected as 60 minutes, 90 minutes, 120 minutes, 150 minutes, 180 minutes, 210 minutes, 240 minutes;
S2. Prepare an in vitro mRNA transcription system and add the solid-phase medium into the transcription system mixture to carry out the transcription reaction for mRNA preparation. The timing of adding the solid-phase medium into the transcription system mixture can be at the transcription initiation stage, during the transcription process, or at the post-transcription stage, preferably at the transcription initiation stage. The amount of the solid-phase medium added is 1-1000 mg/ml, preferably 10-600 mg/ml, and more preferably 10-200 mg/ml, and can be selected as 10 mg/ml, 50 mg/ml, 100 mg/ml, 200 mg/ml. The transcription system mixture includes reaction buffer, DNA template, NTP (Nucleoside Triphosphate.), T7 polymerase, RNase A inhibitor and pyrophosphatase. The transcription reaction temperature is 20-60° C., and can be selected as 20° C., 37° C., 55° C., 60° C., preferably 37° C. The transcription time is 4 hours (0 hour is the transcription initiation stage, and >4 hours is the post-transcription stage). The concentration of the above-mentioned NTP can be 2-8 M. In NTP, ATP (Adenosine Tri-phosphate) includes natural ATP, N1-methyladenosine (m1A), N6-methyladenosine (m6A); CTP (Cytidine Triphosphate) includes natural CTP or 5-methylcytidine (m5C); UTP (Uridine Tri-phosphate) includes natural UTP, 5-methoxyuridine (5moU), pseudouridine (w) or N1-methylpseudouridine (mlv). A cap analogue can also be added to the transcription system in step S2 for co-transcriptional capping. The cap analogues include: CAP GAG, CAP GAG (3′OMe) or CAP GAG (m6A).

S3. After the transcription is completed, collect the supernatant by centrifugation or gravitational sedimentation methods to obtain the mRNA solution. The centrifugation speed is 8000-12000 rpm/min, preferably 10000 rpm/min. The centrifugation time is 1-3 minutes, preferably 2 minutes.

The second inventive point of this application is to provide a kit for in vitro transcription synthesis of mRNA that can reduce or inhibit the formation of dsRNA. The kit adopts the above-mentioned high-yield preparation method of mRNA for reducing or inhibiting the formation of double-stranded ribonucleic acid during the in vitro transcription process. The kit includes solid-phase medium, medium equilibration solution/buffer, transcription reaction solution, positive control DNA, NTP, T7 polymerase, RNase A inhibitor, sterile and nuclease-free water, and pyrophosphatase.

Optionally, in the above-mentioned kit, the solid-phase medium is a medium modified with negative charge groups. The negative charge groups include one or more of sulfonic acid group —SO₃, methylsulfonic acid group —CH₂SO₃, ethylsulfonic acid group —(CH₂)₂SO₃, propylsulfonic acid group —(CH₂)₃SO₃, phosphate group PO₃, carboxylic acid group —COO, formyl group —CH₂COO, hydroxyl group —OH, polyadenylic acid Ploy A, polythymidylic acid Ploy T, polyuridylic acid Ploy U, polyguanylic acid Ploy G and polycytidylic acid Ploy C. Wherein, the degree of polymerization n of Ploy A (polyadenylic acid), Ploy T (polythymidylic acid), Ploy U (polyuridylic acid), Ploy G (polyguanylic acid) and Ploy C (polycytidylic acid) is 1 to 100; and the degrees of polymerization can be selected as 1, 5, 10, 15, 20, 25, 50, 75, 100;
The form of the solid-phase medium is one or more of granular, membranous and sheet-like;
The granular solid-phase medium includes one or more of microspheres and nanoparticles;
The material of the granular solid-phase medium includes one or more of organic materials, inorganic materials and functional materials;
The organic material includes one or more of natural polysaccharides and synthetic polymers;
The natural polysaccharide organic materials include one or more of cellulose, dextran, agarose, chitosan and konjac glucomannan;
The synthetic polymers include one or more of styrenic polymers, acrylic polymers and polyvinyl acid-type polymers;
The inorganic materials include one or more of silica gel, glass, metal oxides and hydroxyapatite;
The functional materials include one or more of magnetic materials and thermos-sensitive materials;
The membranous solid-phase medium includes one or more of nitrocellulose membrane, nylon membrane and glass-cellulose membrane;
The sheet-like solid-phase medium includes carbon nanotubes;
The medium equilibration solution/buffer includes one or more of Tris-HCl buffer, citrate buffer, acetate buffer, phosphate buffer and HEPES buffer.

Preferably, the medium equilibration solution/buffer is Tris-HCl buffer or phosphate buffer.

Preferably, the buffer concentration is 0.01-1M, and can be selected as 0.01M, 0.05M, 0.1M, 0.3M, 0.5M, 0.7M, 1M.

The transcription reaction solution contains a basic buffer, inorganic salts and reducing agents. The basic buffer includes one or more of Tris-HCl buffer, citrate buffer, acetate buffer, phosphate buffer and HEPES buffer. The inorganic salts include one or more of NaCl, KCl, MgCl₂, Na₂SO₄, K₂SO₄, MgSO₄. The reducing agents include one or more of DTT, mercaptoethanol, and reduced glutathione.

The third inventive point of this invention is to provide a method of using the above-mentioned kit, which includes the following steps:
T1. Take the solid-phase medium from the kit for pretreatment. The pretreatment involves washing the solid-phase medium with sterile and nuclease-free water and then equilibrating it with the medium equilibration solution/buffer;
T2. Use the reagents in the kit to prepare an in vitro mRNA transcription system, and add the pretreated solid-phase medium into the transcription system mixture to carry out the transcription reaction for mRNA preparation;
T3. After the transcription is completed, collect the supernatant by centrifugation or gravitational sedimentation methods to obtain the mRNA solution.

Optionally, in the above-mentioned method of using the kit, in step T1, the pretreatment is to wash the solid-phase medium with sterile and nuclease-free water, then equilibrate it with the medium equilibration solution, and drain it after shaking for equilibration. The shaking equilibration time is 5-240 minutes. Preferably, the shaking equilibration time is preferably 60-240 minutes and can be selected as 60 minutes, 90 minutes, 120 minutes, 150 minutes, 180 minutes, 210 minutes, 240 minutes; in step T2, for the pretreated solid-phase medium, the timing of adding it into the transcription system mixture can be at the transcription initiation stage, during the transcription process, or at the post-transcription stage, preferably at the transcription initiation stage. The amount of the solid-phase medium added is 1-1000 mg/ml, preferably 10-200 mg/ml. The transcription system mixture includes transcription reaction solution, DNA template, NTP, T7 polymerase, RNase A inhibitor and pyrophosphatase. The transcription reaction temperature is 20-60° C.

The amount of the solid-phase medium added is 1-1000 mg/ml, preferably 10-600 mg/ml, more preferably 10-200 mg/ml, and can be selected as 10 mg/ml, 50 mg/ml, 100 mg/ml, 200 mg/ml.

The transcription reaction temperature is 20-60° C., and can be selected as 20° C., 37° C., 55° C., 60° C., preferably 37° C. The transcription time is 4 hours (0 hour is the transcription initiation stage, and >4 hours is the post-transcription stage).

The concentration of NTP can be 2-8 M. In NTP, ATP includes natural ATP, N1-methyladenosine (m1A), N6-methyladenosine (m6A); CTP includes natural CTP or 5-methylcytidine (m5C); UTP includes natural UTP, 5-methoxyuridine (5moU), pseudouridine (v) or N1-methylpseudouridine (ml).

The transcription system in step T2 can also have a cap analogue added for co-transcriptional capping. The cap analogues include: CAP GAG, CAP GAG (3′OMe) or CAP GAG (m6A).

Compared with the prior art, this application has the following advantages:

The high-yield preparation method and kit for mRNA that reduce or inhibit the formation of double-stranded ribonucleic acid during in vitro transcription provided by this invention involve adding various types of solid-phase media with negative charges in the in vitro transcription process. Through interface regulation, the generation of dsRNA is reduced, while the yield and stability of mRNA are enhanced. Moreover, the transfection efficiency of mRNA prepared by solid-phase control is improved, and the expression of immune factors is decreased. The solid-phase media used in this method and kit are insoluble in water and will not contaminate the transcription system. After the transcription is completed, the solid-phase media can be easily separated, and the operation is simple. The solid-phase media can be reused after appropriate treatment, with low cost and being easy to scale up to industrial-scale production.

### BRIEF DESCRIPTION OF DRAWINGS

FIG. 1 shows the HPLC detection spectra of Example 3-1, Comparative Example 1 and Comparative Example 2.

FIG. 2 shows the relative mRNA yields calculated based on the peak areas detected by HPLC.

FIG. 3 shows the mRNA yields detected by agarose gel electrophoresis for Example 3-1, Comparative Example 1 and Comparative Example 2.

FIG. 4 shows the dsRNA yields detected by dot blot for Example 3-1, Comparative Example 1 and Comparative Example 2.

FIG. 5 shows the HPLC detection spectra of Example 3-1 and Comparative Example 4.

FIG. 6 shows the mRNA yields detected by agarose gel electrophoresis for Example 3-1 and Comparative Example 4.

FIG. 7 shows the dsRNA yields detected by dot blot for Example 3-1 and Comparative Example 4.

FIG. 8 shows the transfection efficiency of the mRNA prepared by the method of Example 20 as measured in Example 21.

FIG. 9 shows the IFN-β levels of the cells transfected with the mRNA prepared by the method of Example 20 as measured in Example 21.

### DETAILED DESCRIPTION

To make the purpose, technical solutions, and advantages of this application clearer and more explicit, the following provides a further detailed explanation of this application. However, it should be understood that the description here is merely used to explain this application and is not intended to limit the scope of this application.

Unless otherwise defined, all technical and scientific terms used herein have the same meanings as those commonly understood by technicians in the technical field to which this application pertains. The terms used in the specification of this application are only for the purpose of describing specific embodiments and are not intended to limit this application. The reagents and instruments used herein are all commercially available, and the characterization methods involved can be referred to the relevant descriptions in the prior art, and will not be elaborated herein.

To further understand this application, the following provides a more detailed explanation of this application in combination with the best embodiments.

### Example 1

A high-yield preparation method for mRNA that reduces or inhibits the formation of double-stranded ribonucleic acid during in vitro transcription. The preparation method involves adding a solid-phase medium during the transcription process.

The solid-phase medium is a medium modified with negatively charged groups.

This modification can be at any position of the solid-phase medium. For example, negatively charged groups can be modified on the surface of the solid-phase medium, or negatively charged groups can be modified inside the solid-phase medium, etc.

The negatively charged groups modified on the solid-phase medium are one or more of sulfonic acid group —SO₃, methylsulfonic acid group —CH₂SO₃, ethylsulfonic acid group —(CH₂)₂SO₃, propylsulfonic acid group —(CH₂)₃SO₃, phosphate group PO₃, carboxylic acid group —COO, formyl group —CH₂COO, hydroxyl group —OH, polyadenylic acid Ploy A, polythymidylic acid Ploy T, polyuridylic acid Ploy U, polyguanylic acid Ploy G, and polycytidylic acid Ploy C.

Wherein, the degree of polymerization (n) of Ploy A (polyadenylic acid), Ploy T (polythymidylic acid), Ploy U (polyuridylic acid), Ploy G (polyguanylic acid), and Ploy C (polycytidylic acid) is 1 to 100; and the degrees of polymerization and selections are 1, 5, 10, 15, 20, 25, 50, 75, and 100.

The form of the solid-phase medium is one or more of granular, membranous and sheet-like.

The granular solid-phase medium includes one or more of microspheres and nanoparticles;
The material of the granular solid-phase medium includes one or more of organic materials, inorganic materials and functional materials;
The organic material includes one or more of natural polysaccharides and synthetic polymers;
The natural polysaccharide organic materials include one or more of cellulose, dextran, agarose, chitosan and konjac glucomannan;
The synthetic polymers include one or more of styrenic polymers, acrylic polymers and polyvinyl acid-type polymers;
The inorganic materials include one or more of silica gel, glass, metal oxides and hydroxyapatite;
The functional materials include one or more of magnetic materials and thermos-sensitive materials;
The membranous solid-phase medium includes one or more of nitrocellulose membrane, nylon membrane and glass-cellulose membrane;
The sheet-like solid-phase medium includes carbon nanotubes.

The preparation method comprises the following steps:
S1. Pretreatment of Solid-Phase Medium: washing the solid-phase medium with sterile and nuclease-free water, then equilibrate it with a buffer solution. After shaking for equilibration, drain it by suction. Preferably, the buffer solution includes one or more of Tris-HCl buffer, citrate buffer, acetate buffer, phosphate buffer and HEPES buffer. The shaking equilibration time is 5-240 minutes. The equilibrated solid-phase medium is drained by suction through a sintered glass filter. The buffer solution is preferably Tris-HCl buffer or phosphate buffer. The concentration of the buffer solution is 0.01-1 M, and can be selected as 0.01 M, 0.05 M, 0.1 M, 0.3 M, 0.5 M, 0.7 M, 1 M. The shaking equilibration time is preferably 60-240 minutes, and can be selected as 60 minutes, 90 minutes, 120 minutes, 150 minutes, 180 minutes, 210 minutes, 240 minutes;
S2. Preparing an in vitro mRNA transcription system and adding the solid-phase medium into the transcription system mixture to carry out the transcription reaction for mRNA preparation. The timing of adding the solid-phase medium into the transcription system mixture can be at the transcription initiation stage, during the transcription process, or at the post-transcription stage, preferably at the transcription initiation stage. The amount of the solid-phase medium added is 1-1000 mg/ml, preferably 10-600 mg/ml, and more preferably 10-200 mg/ml, and can be selected as 10 mg/ml, 50 mg/ml, 100 mg/ml, 200 mg/ml. The transcription system mixture includes reaction buffer, DNA template, NTP (Nucleoside Triphosphate.), T7 polymerase, RNase A inhibitor and pyrophosphatase. The transcription reaction temperature is 20-60° C., and can be selected as 20° C., 37° C., 55° C., 60° C., preferably 37° C. The transcription time is 4 hours (0 hour is the transcription initiation stage, and >4 hours is the post-transcription stage). The concentration of the above-mentioned NTP can be 2-8 M. In NTP, ATP (Adenosine Tri-phosphate) includes natural ATP, N1-methyladenosine (m1A), N6-methyladenosine (m6A); CTP (Cytidine Triphosphate) includes natural CTP or 5-methylcytidine (m5C); UTP (Uridine Tri-phosphate) includes natural UTP, 5-methoxyuridine (5moU), pseudouridine (w) or N1-methylpseudouridine (mlw). A cap analogue can also be added to the transcription system for co-transcriptional capping. The cap analogues include: CAP GAG, CAP GAG (3′OMe) or CAP GAG (m6A).

S3. After the transcription is completed, collecting the supernatant by centrifugation or gravitational sedimentation methods to obtain the mRNA solution. The centrifugation speed is 8000-12000 rpm/min, preferably 10000 rpm/min. The centrifugation time is 1-3 minutes, preferably 2 minutes.

This application also provides a kit for in vitro transcription synthesis of mRNA that can reduce or inhibit the formation of dsRNA. The kit adopts the above-mentioned high-yield preparation method of mRNA for reducing or inhibiting the formation of double-stranded ribonucleic acid during the in vitro transcription process. The kit includes solid-phase medium, medium equilibration solution/buffer, transcription reaction solution, positive control DNA, NTP, T7 polymerase, RNase A inhibitor, sterile and nuclease-free water, and pyrophosphatase.

In the kit, the solid-phase medium is a medium modified with negative charge groups. The negative charge groups include one or more of sulfonic acid group —SO₃, methylsulfonic acid group —CH₂SO₃, ethylsulfonic acid group —(CH₂)₂SO₃, propylsulfonic acid group —(CH₂)₃SO₃, phosphate group PO₃, carboxylic acid group —COO, formyl group —CH₂COO, hydroxyl group —OH, polyadenylic acid Ploy A, polythymidylic acid Ploy T, polyuridylic acid Ploy U, polyguanylic acid Ploy G and polycytidylic acid Ploy C. Wherein, the degree of polymerization n of Ploy A (polyadenylic acid), Ploy T (polythymidylic acid), Ploy U (polyuridylic acid), Ploy G (polyguanylic acid) and Ploy C (polycytidylic acid) is 1 to 100; and the degrees of polymerization can be selected as 1, 5, 10, 15, 20, 25, 50, 75, 100;
The form of the solid-phase medium is one or more of granular, membranous and sheet-like;
The granular solid-phase medium includes one or more of microspheres and nanoparticles;
The material of the granular solid-phase medium includes one or more of organic materials, inorganic materials and functional materials;
The organic material includes one or more of natural polysaccharides and synthetic polymers;
The natural polysaccharide organic materials include one or more of cellulose, dextran, agarose, chitosan and konjac glucomannan;
The synthetic polymers include one or more of styrenic polymers, acrylic polymers and polyvinyl acid-type polymers;
The inorganic materials include one or more of silica gel, glass, metal oxides and hydroxyapatite;
The functional materials include one or more of magnetic materials and thermos-sensitive materials;
The membranous solid-phase medium includes one or more of nitrocellulose membrane, nylon membrane and glass-cellulose membrane;
The sheet-like solid-phase medium includes carbon nanotubes;
The medium equilibration solution/buffer includes one or more of Tris-HCl buffer, citrate buffer, acetate buffer, phosphate buffer and HEPES buffer.

Preferably, the medium equilibration solution/buffer is Tris-HCl buffer or phosphate buffer.

Preferably, the buffer concentration is 0.01-1M, and can be selected as 0.01M, 0.05M, 0.1M, 0.3M, 0.5M, 0.7M, 1M.

The transcription reaction solution contains a basic buffer, inorganic salts and reducing agents. The basic buffer includes one or more of Tris-HCl buffer, citrate buffer, acetate buffer, phosphate buffer and HEPES buffer. The inorganic salts include one or more of NaCl, KCl, MgCl₂, Na₂SO₄, K₂SO₄, MgSO₄. The reducing agents include one or more of DTT, mercaptoethanol, and reduced glutathione.

This application also provides a method of using the above-mentioned kit, which includes the following steps:
T1. Take the solid-phase medium from the kit for pretreatment. The pretreatment involves washing the solid-phase medium with sterile and nuclease-free water and then equilibrating it with the medium equilibration solution/buffer;
T2. Use the reagents in the kit to prepare an in vitro mRNA transcription system, and add the pretreated solid-phase medium into the transcription system mixture to carry out the transcription reaction for mRNA preparation;
T3. After the transcription is completed, collect the supernatant by centrifugation or gravitational sedimentation methods to obtain the mRNA solution.

In step T1, the pretreatment is to wash the solid-phase medium with sterile and nuclease-free water, then equilibrate it with the medium equilibration solution, and drain it after shaking for equilibration. The shaking equilibration time is 5-240 minutes. Preferably, the shaking equilibration time is preferably 60-240 minutes and can be selected as 60 minutes, 90 minutes, 120 minutes, 150 minutes, 180 minutes, 210 minutes, 240 minutes; in step T2, for the pretreated solid-phase medium, the timing of adding it into the transcription system mixture can be at the transcription initiation stage, during the transcription process, or at the post-transcription stage, preferably at the transcription initiation stage. The amount of the solid-phase medium added is 1-1000 mg/ml, preferably 10-200 mg/ml. The transcription system mixture includes transcription reaction solution, DNA template, NTP, T7 polymerase, RNase A inhibitor and pyrophosphatase. The transcription reaction temperature is 20-60° C.

The amount of the solid-phase medium added is 1-1000 mg/ml, preferably 10-600 mg/ml, more preferably 10-200 mg/ml, and can be selected as 10 mg/ml, 50 mg/ml, 100 mg/ml, 200 mg/ml.

The transcription reaction temperature is 20-60° C., and can be selected as 20° C., 37° C., 55° C., 60° C., preferably 37° C. The transcription time is 4 hours (0 hour is the transcription initiation stage, and >4 hours is the post-transcription stage).

The concentration of NTP can be 2-8 M. ATP in NTP includes natural ATP, N1-methyladenosine (m1A), N6-methyladenosine (m6A); CTP includes natural CTP or 5-methylcytidine (m5C); UTP includes natural UTP, 5-methoxyuridine (5moU), pseudouridine (v), or N1-methylpseudouridine (mlv).

The transcription system in step T2 can also have a cap analogue added for co-transcriptional capping. The cap analogues include: CAP GAG, CAP GAG (3′OMe) or CAP GAG (m6A).

### Example 2

A high-yield preparation method of mRNA, comprising the following steps:

### S1. Pretreatment of Solid-Phase Medium:

Wash the agarose microspheres modified with sulfonic acid group —SO₃ using sterile and nuclease-free water, and then wash and equilibrate them with Tris-HCl buffer solution with a concentration of 0.05 M prepared by sterile and nuclease-free water. The equilibration time is 240 minutes. Before use, settle and separate them by centrifugation or under the action of gravity;

S2. In vitro transcription of mRNA:

Prepare an in vitro mRNA transcription system, and add the solid-phase medium into the transcription system mixture at the initial stage of the transcription reaction, that is, at the 0 h stage of the reaction, to prepare mRNA by the transcription reaction. Among them, the amount of the solid-phase medium added is 100 mg/ml. The preparation method of the mRNA transcription system (reaction system of 100 μL) is as follows:
(1) Prepare 10× Transcription Buffer: 400 mM Tris-HCl with pH 7.9, 60 mM MgCl₂, 100 mM DTT (dithiothreitol), 20 mM spermidine;
(2) Place the required T7 RNA Polymerase, RNase inhibitor, and pyrophosphatase on ice; mix ATP, CTP, GTP, and UTP (UTP modified with pseudouridine, UTP modified with N1-methylpseudouridine, CTP modified with 5-methylcytidine, ATP modified with N1-methyladenosine) evenly and place them on ice. Sterile and nuclease-free water, DNA template, and 10× Transcription Buffer are placed at room temperature for later use (if it is a co-transcription capping system, the cap analogues are CAP GAG, CAP GAG (3′OMe), or CAP GAG (m6A));
(3) Add 10 μL of 10× Transcription Buffer, 41.5 μL of RNase Free Water, 8 μL of NTP (nucleoside triphosphate), 30 μL of DNA template, 5 μL of T7 RNA Polymerase, 4 μL of RNase inhibitor, and 1.5 μL of pyrophosphatase (add 2 μL of cap analogue if it is a co-transcription capping system) into a sterile and nuclease-free centrifuge tube. Gently mix the components and incubate at 37° C. for 4 hours;
S3. After the transcription is completed, the supernatant is collected by centrifugation or gravitational sedimentation to obtain the mRNA solution.

In the kit, specifically:

Wash the agarose-dextran microspheres modified with sulfonic acid group —SO3 using sterile and nuclease-free water, and then equilibrate them with Tris-HCl medium equilibration buffer solution with a concentration of 0.05M prepared by sterile and nuclease-free water. The equilibration time is 240 minutes. Before use, settle and separate them by centrifugation or under the action of gravity.

At the initial stage of the transcription reaction, that is, at the 0-hour stage of the reaction, add the above pre-equilibrated solid-phase medium into the transcription system mixture. The reaction system is 100 μL, and mRNA is prepared by the transcription reaction. The specific amount of each component added in the kit is as follows:

1) The amount of the solid-phase medium added is 100 mg/ml.

2) 10 μL of 10× Transcription Buffer: 400 mM Tris-HCl with pH 7.9, 60 mM MgCl₂, 100 mM DTT (dithiothreitol), 20 mM spermidine.

3) 30 μL of green fluorescent protein DNA template.

4) 2 μL each of ATP, CTP, GTP, and UTP (add 2 μL of cap analogue if it is a co-transcription capping system).

5) 4 μL of RNase inhibitor and 1.5 μL of pyrophosphatase.

6) The amount of T7 RNA Polymerase added is 5 μL.

7) Make up to 100 μL with sterile and nuclease-free water.

Gently mix the above components and incubate at 37° C. for 4 hours. After the transcription is completed, collect the supernatant by centrifugation or gravitational sedimentation to obtain the mRNA solution.

### Example 3

Different Kinds of Solid-Phase Media Modified with Sulfonic Acid Groups are Added In Vitro Transcription

A method for reducing the formation of dsRNA during in vitro transcription for preparing mRNA, with the specific steps as follows:

It is substantially the same as the method described in Example 2, with the difference that the sulfonic acid group-modified solid-phase media added are different. Five different sulfonic acid group-modified solid-phase media with a dosage of 5 mg each are added to the transcription system, and five 100 μL transcription mixtures with the gene of green fluorescent protein as the template are prepared respectively.

In using the kit, the previous kit and method are used. The difference is that the matrices of the solid-phase media in the kit are different, namely sulfonic acid group-modified agarose microspheres, nitrocellulose membrane, silica matrix, nanofibers, and magnetic microspheres. 5 mg of each of the 5 different sulfonic acid group-modified solid-phase media are added to the transcription system for transcription with the gene of green fluorescent protein as the template.

Table 1 shows the five different sulfonic acid group-modified solid-phase media selected for use.

TABLE 1

| Serial Number | 1 | 2 | 3 | 4 | 5 |
|---|---|---|---|---|---|
| Types of Solid - | Agarose | Nitrocellulose | Silica | Nanofibers | Magnetic |
| Phase Media | Microspheres | Membrane | Matrix | | Microspheres |

Gently mixing each component and incubating at 37° C. for 4 hours. After transcription is completed, separating the transcription mixture and the solid-phase media.

### Example 4

Add Agarose Microsphere Media Modified with Different Groups In Vitro Transcription:

A method for reducing the formation of dsRNA during the process of in vitro transcription for preparing mRNA, with the specific steps as follows:

The method is basically the same as that described in Example 2. The difference lies in that the added dextran microsphere solid-phase media are modified with five different groups. 10 mg of dextran microsphere solid-phase media modified with five different negatively charged groups are added to the transcription system, and five 100 μL transcription mixtures with the gene of green fluorescent protein as the template are prepared respectively.

During the use of the kit, it is roughly the same as that described in Example 2. The difference lies in that the solid-phase medium in the kit is agarose microspheres modified with five different groups. 10 mg of dextran microsphere solid-phase media modified with five different negatively charged groups are added to the transcription system respectively, and transcription is carried out with the gene of green fluorescent protein as the template.

Table 2 shows the five different negatively charged groups used for surface modification of the selected dextran microsphere solid-phase media.

TABLE 2

| Serial Number | 1 | 2 | 3 | 4 | 5 | 6 |
|---|---|---|---|---|---|---|
| modification | carboxylic | sulfonic | phosphate | formyl | polycytosine | Polythymine |
| group | acid group | acid group | group | group | (n = 25) | (n = 25) |

Gently mix each component and incubate at 37° C. for 4 hours. After the transcription is completed, centrifuge at 12,000 rpm for 2 minutes to separate the transcription mixture and the microspheres. When the solid-phase medium modified with polythymine is used, a small amount of the transcriptionally synthesized mRNA will be adsorbed. You can add 100 μL of 20 mM Tris-HCl+1M NaCl (pH 7.0) to the microspheres for rinsing, and then centrifuge at 12,000 rpm for 2 minutes to separate the transcription mixture and the microspheres again. Next, add 100 μL of sterile and nuclease-free water to the microspheres, place them on a shaker, and elute at 180 r/min at 60° C. for 1 hour. Finally, centrifuge at 12,000 rpm for 2 minutes to separate the elution sample and the microspheres, so that the small amount of mRNA adsorbed on the medium can be retrieved.

### Example 5

### In Vitro Transcription of Solid-Phase Media at Different Concentrations

A method for reducing the formation of dsRNA during in vitro transcription to prepare mRNA, the specific steps are as follows:

The method is basically the same as that described in Example 2. The difference lies in the different concentrations (addition amounts) of the dextran-agarose solid-phase medium modified with sulfonic acid group. The dextran-agarose solid-phase medium modified with sulfonic acid group is added into the transcription system at six different concentration addition amounts respectively to prepare six 100 μL transcription mixtures with the gene of green fluorescent protein as the template.

During the use of the kit, it is roughly the same as described in Example 2. The difference lies in the different concentrations (addition amounts) of the dextran-agarose solid-phase medium modified with sulfonic acid group. The dextran-agarose solid-phase medium modified with sulfonic acid group is added into the transcription system at six different concentration addition amounts respectively, and the transcription is carried out using the gene of green fluorescent protein as the template.

Table 3 shows the six different addition amounts of the dextran-agarose solid-phase medium modified with sulfonic acid group that are selected and used.

TABLE 3

| Serial Number | 1 | 2 | 3 | 4 | 5 | 6 |
|---|---|---|---|---|---|---|
| The addition amount of solid- | 10 | 50 | 100 | 200 | 400 | 600 |
| phase medium (mg/mL) | | | | | | |

Gently mix each component and incubate at 37° C. for 4 hours. After the transcription is completed, centrifuge at 12,000 rpm for 2 minutes to separate the transcription mixture and the microspheres.

### Example 6

### Add the Solid-Phase Medium at Different Times During In Vitro Transcription:

A method for reducing the formation of double-stranded RNA (dsRNA) during the process of in vitro transcription to prepare mRNA. The specific steps are as follows:

The method is basically the same as that described in Example 2. The difference lies in the fact that the 5 mg agarose microsphere solid-phase medium modified with sulfonic acid group is added at different times. The already equilibrated solid-phase medium is added to the transcription system at four different times to prepare four 100 μL transcription mixtures with the gene of green fluorescent protein as the template.

During the use of the kit, it is basically the same as what is described in Example 2. The difference is that the 5 mg agarose microsphere solid-phase medium modified with sulfonic acid group is added at different times after the start of transcription. The already equilibrated solid-phase medium is added to the transcription system at four different times to conduct the transcription reaction with the gene of green fluorescent protein as the template.

Table 4 shows the four different addition timings of the solid-phase medium during the in vitro transcription process that are selected and used.

TABLE 4

| Serial Number | 1 | 2 | 3 | 4 |
|---|---|---|---|---|
| Transcription time (h) | 0 | 1 | 2 | >4 |

Gently mix each component and incubate them together at 37° C. for 4 hours. After the transcription is completed, separate the transcription mixture and the microspheres.

### Example 7

Add Solid-Phase Media Modified with Groups of Different Polymerization Lengths During In Vitro Transcription:

A method for reducing the formation of dsRNA during the process of in vitro transcription to prepare mRNA. The specific steps are as follows:

The method is basically the same as that described in Example 2. The difference lies in the fact that the 10 mg solid-phase medium added to the transcription system is the solid-phase medium (polystyrene matrix) modified with polythymine of four different degrees of polymerization. Four 100 μL transcription mixtures with the gene of green fluorescent protein as the template are prepared.

During the use of the kit, it is approximately the same as what is described in Example 2. The difference is that the 10 mg solid-phase medium added to the transcription system is the solid-phase medium (polystyrene matrix) modified with polythymine of four different degrees of polymerization. In vitro transcription is carried out respectively using the genes of foot-and-mouth disease proteins as the templates.

Table 5 shows the four different degrees of polymerization of the solid-phase medium (polystyrene matrix) modified with polythymine that are selected and used.

TABLE 5

| Serial Number | 1 | 2 | 3 | 4 |
|---|---|---|---|---|
| degree of | 10 | 18 | 25 | 50 |
| polymerization (n) | | | | |

Gently mix each component and incubate at 37° C. for 4 hours. After the transcription is completed, centrifuge at 12,000 rpm for 2 minutes to separate the transcription mixture and the microspheres. For this type of medium, a small amount of the transcription-synthesized mRNA will be adsorbed. You can add 100 μL of 20 mM Tris-HCl+1M NaCl (pH 7.0) to the microspheres for rinsing, and then centrifuge at 12,000 rpm for 2 minutes to separate the transcription mixture and the microspheres again. Next, add 100 μL of sterile and nuclease-free water to the microspheres, place them on a shaker, and elute at 180 r/min at 60° C. for 1 hour. Finally, centrifuge at 12,000 rpm for 2 minutes to separate the eluted sample and the microspheres, so that the small amount of mRNA adsorbed on the medium can be recovered.

### Example 8

### Different Transcription Temperatures in the In Vitro Transcription System:

A method for reducing the formation of dsRNA during the process of in vitro transcription to prepare mRNA. The specific steps are as follows:

The method is basically the same as that described in Example 2. The difference lies in the fact that after adding 5 mg of agarose matrix microsphere solid-phase medium modified with carboxylic acid groups to the transcription system, in vitro transcription is carried out at three different temperatures to prepare three 100 μL transcription mixtures with the gene of green fluorescent protein as the template.

During the use of the kit, it is approximately the same as what is described in Example 2. The difference is that after adding 5 mg of agarose matrix microsphere solid-phase medium modified with carboxylic acid groups to the transcription system, in vitro transcription is carried out at three different temperatures.

Table 6 shows the three different in vitro transcription temperatures that are selected and used.

TABLE 6

| Serial Number | 1 | 2 | 3 |
|---|---|---|---|
| Temperature (° C.) | 20 | 37 | 55 |

After the incubation for 4 hours and the completion of transcription, centrifuge at 12,000 rpm for 2 minutes to separate the transcription mixture and the microspheres.

### Example 9

### Add Solid-Phase Media in the In Vitro Transcription of Differently Modified NTPs:

A method for reducing the formation of dsRNA during the process of in vitro transcription to prepare mRNA. The specific steps are as follows:

The method is basically the same as that described in Example 2. The difference lies in the fact that the NTPs added to the transcription system have undergone four different modifications, and four 100 μL transcription mixtures with the gene of green fluorescent protein as the template are prepared.

During the use of the kit, the method is approximately the same as that described in Example 2. The difference is that the NTPs added to the transcription system have undergone four different modifications.

Table 7 shows the four different NTP modifications selected and used in the in vitro transcription.

TABLE 7

| | NTP | | | |
|---|---|---|---|---|
| | UTP | | CTP | ATP |
| | Serial Number | | | |
| | 1 | 2 | 3 | 4 |
| modification | Pseudo- | N1-Methyl- | 5-Methyl- | N1-Methyl- |
| | uridine | pseudouridine | cytidine | adenosine |
| | (ψ) | (m1ψ) | | |

Add 5 mg of carboxylic acid-modified dextran microspheres to the transcription system respectively, gently mix each component, and incubate at 37° C. for 4 hours. After the transcription is completed, centrifuge at 12,000 rpm for 2 minutes to separate the transcription mixture and the microspheres.

### Example 10

### Reuse the Solid-Phase Medium in the Transcription System:

A method for reducing the formation of dsRNA during the process of in vitro transcription to prepare mRNA. The specific steps are as follows:

The method is basically the same as that described in Example 2. The difference is that a 100 μL transcription mixture with the gene of green fluorescent protein as the template is prepared. Then, 5 mg of agarose-dextran microsphere medium with sulfonic acid groups is added, and each component is gently mixed. Incubate at 37° C. for 4 hours. After the transcription is completed, centrifuge at 12,000 rpm for 2 minutes to separate the transcription mixture and the microspheres. Add 100 μL of 20 mM Tris-HCl (pH 7.0) to the microspheres for rinsing, and centrifuge at 12,000 rpm for 2 minutes to separate the transcription mixture and the microspheres again. Then, add 100 μL of 20 mM Tris-HCl+1M NaCl (pH 7.0) to the microspheres, place it on a shaker, and elute at 180 r/min at 60° C. for 1 hour. Finally, centrifuge at 12,000 rpm for 2 minutes to separate the eluted sample and the microspheres.

The eluted microspheres are re-equilibrated in a 20 mM Tris-HCl (pH 7.0) buffer, and the microspheres are reused twice following the above steps.

In the use of the kit, the difference from what is described in Example 2 lies in the following: Prepare a 100 μL transcription mixture with the gene of green fluorescent protein as the template, add 5 mg of agarose microsphere medium with sulfonic acid groups, gently mix each component, and incubate at 37° C. for 4 hours. After the transcription is completed, centrifuge at 12,000 rpm for 2 minutes to separate the transcription mixture and the microspheres.

Add 100 μL of 20 mM Tris-HCl (pH 7.0) to the microspheres for rinsing, and centrifuge at 12,000 rpm for 2 minutes to separate the transcription mixture and the microspheres. Then, add 100 μL of 20 mM Tris-HCl+1M NaCl (pH 7.0) to the microspheres, place them on a shaker, and elute at 180 r/min at 60° C. for 1 hour. Centrifuge at 12,000 rpm for 2 minutes to separate the eluted sample and the microspheres.

Re-equilibrate the eluted microspheres in a 20 mM Tris-HCl (pH 7.0) buffer, and reuse the microspheres twice according to the above steps.

Table 8 shows the number of times the solid-phase medium is used during the in vitro transcription process.

TABLE 8

| Serial Number | 1 | 2 | 3 |
|---|---|---|---|
| Number of times the solid-phase | 1 | 2 | 3 |
| medium is used (times) | | | |

### Example 11

### Mixed Use of Multiple Solid-Phase Media:

A method for reducing the formation of dsRNA during the process of in vitro transcription to prepare mRNA. The specific steps are as follows:

The method is basically the same as that described in Example 2. The difference lies in the fact that a mixture of solid-phase media with three different modifications is added to the transcription system, and three 100 μL transcription mixtures with the gene of green fluorescent protein as the template are prepared.

During the use of the kit, it is approximately the same as that described in Example 2. The difference is that a mixture of solid-phase media with three different modifications is added to the transcription system.

Table 9 shows the three different mixtures of solid-phase media selected and used in the in vitro transcription.

TABLE 9

| Serial | | |
|---|---|---|
| Number | solid - phase media 1 | solid - phase media 2 |
| 1 | 5 mg carboxylic - acid - | 5 mg Phosphate - group - |
| | modified agarose | modified dextran |
| | microspheres | microspheres |
| 2 | 5 mg Sulfonic - acid - | 5 mg Formic - acid - |
| | modified silica | modified silica |
| | microspheres | microspheres |
| 3 | 5 mg polythymine | 5 mg polythymine |
| | (n = 18, | (n = 25, |
| | Polystyrene matrix) | Polystyrene matrix ) |

Gently mix each component and incubate at 37° C. for 4 hours. After the transcription is completed, centrifuge at 12,000 rpm for 2 minutes to separate the transcription mixture and the solid-phase medium.

### Comparative Example 1

In Comparative Example 1, no solid-phase medium is added to the transcription system.

For the transcription system without adding the solid-phase medium, the operation process is as follows: Take 100 μL of the transcription mixture with the gene of green fluorescent protein as the template, gently mix each component, and incubate at 37° C. for 4 hours.

### Comparative Example 2

In Comparative Example 2, agarose microspheres without ligand modification are added to the transcription system. The difference from Group 1 of Example 3 (where agarose microspheres modified with sulfonic acid groups are added) lies in that the surface of the agarose microspheres without ligand modification is not modified with negative charge groups.

The operation process for the transcription system with agarose microspheres without ligands added is as follows: Take 100 μL of the transcription mixture with the gene of green fluorescent protein as the template, add 5 mg of agarose matrix microspheres without ligands, gently mix each component, and incubate at 37° C. for 4 hours. After the transcription is completed, centrifuge at 12,000 rpm for 2 minutes to separate the transcription mixture and the microspheres. Add 100 μL of 20 mM Tris-HCl (pH 7.0) to the microspheres for rinsing, and then centrifuge at 12,000 rpm for 2 minutes to separate the transcription mixture and the microspheres again. Then add 100 μL of 20 mM Tris-HCl+1M NaCl (pH 7.0) to the microspheres, place them on a shaker, and elute at 180 r/min at 60° C. for 1 hour. Finally, centrifuge at 12,000 rpm for 2 minutes to separate the eluted sample and the microspheres.

### Comparative Example 3

In Comparative Example 3, agarose microspheres modified with positive charge groups are added to the transcription system. The difference from Group 1 of Example 3 (where agarose microspheres modified with sulfonic acid groups are added) lies in that the groups modified on the agarose microspheres added in this comparative example carry positive charges.

### Example 12 (Test Example 1)

The agarose gel electrophoresis method and HPLC method are used to quantify the mRNA in the transcription samples, and the Dot blot method is used to quantitatively analyze the dsRNA in the in vitro transcription system. The yields of mRNA and the contents of dsRNA obtained from Examples 3-11 are calculated, and the results are listed in Table 10.

$$The relative yield of mRNA ( \% ) = the mRNA concentration of the group with solid - phase medium added / the mRNA concentration of the group without solid - phase medium added * 100 \% .$$

$$The relative yield of dsRNA ( \% ) = the content of dsRNA in the mRNA of the group with solid \text{} - phase medium added / the consent of dsRNA in the mRNA of the group without solid - phase medium added * 100 \% .$$

Table 10 shows the comparison of the statistical results of the relative yields of mRNA (%) and the relative yields of dsRNA (%) obtained from Comparative Examples 1-3 and Examples 3-11. Among them, the first item in Table 1 of Example 3 is represented as “Example 3-1”, the second item in Table 1 of Example 3 is represented as “Example 3-2”, and so on for the rest.

TABLE 10

| | The relative yield | The relative yield |
|---|---|---|
| Process | of mRNA (%) | of dsRNA (%) |
| Comparative example 1 | 100 | 100 |
| Comparative example 2 | 98.97 | 82.56 |
| Comparative example 3 | 3.48 | 95.35 |
| Example 3-1 | 165.79 | 5.12 |
| Example 3-2 | 148.77 | 7.33 |
| Example 3-3 | 150.36 | 7.68 |
| Example 3-4 | 145.98 | 6.87 |
| Example 3-5 | 161.87 | 8.45 |
| Example 4-1 | 143.57 | 10.87 |
| Example 4-2 | 170.12 | 5.02 |
| Example 4-3 | 160.23 | 5.89 |
| Example 4-4 | 152.25 | 7.41 |
| Example 4-5 | 124.82 | 43.25 |
| Example 4-6 | 120.45 | 50.45 |
| Example 5-1 | 135.89 | 12.97 |
| Example 5-2 | 159.72 | 5.22 |
| Example 5-3 | 95.92 | 3.32 |
| Example 5-4 | 85.98 | 1.45 |
| Example 5-5 | 81.63 | 1.23 |
| Example 5-6 | 76.84 | 1.03 |
| Example 6-1 | 165.79 | 5.12 |
| Example 6-2 | 146.72 | 8.41 |
| Example 6-3 | 128.54 | 56.23 |
| Example 6-4 | 110.26 | 72.45 |
| Example 7-1 | 140.89 | 40.73 |
| Example 7-2 | 145.12 | 35.53 |
| Example 7-3 | 148.89 | 35.73 |
| Example 7-4 | 157.89 | 24.73 |
| Example 8-1 | 153.26 | 9.21 |
| Example 8-2 | 142.77 | 9.47 |
| Example 8-3 | 139.26 | 8.91 |
| Example 9-1 | 150.26 | 3.81 |
| Example 9-2 | 162.26 | 2.27 |
| Example 9-3 | 143.26 | 4.91 |
| Example 9-4 | 148.26 | 5.81 |
| Example 10-1 | 159.72 | 5.22 |
| Example 10-2 | 161.72 | 5.72 |
| Example 10-3 | 160.79 | 5.39 |
| Example 11-1 | 138.91 | 5.02 |
| Example 11-2 | 142.91 | 4.85 |
| Example 11-3 | 135.01 | 19.87 |

### Example 13 (Test Example 2)

HPLC tests are carried out on Group 1 of Example 3 (Example 3-1), as well as on Comparative Example 1 and Comparative Example 2. The samples after transcription are diluted times and then subjected to quantitative analysis by HPLC.

mRNA Quantitative Testing Method: The samples after transcription are subjected to HPLC quantitative analysis on an Arc HPLC series (Waters, USA) using an SEC-2000 (300×7.8 mm) analytical column (Sepax, USA), with the ultraviolet detection wavelength set at 260 nm. In each operation, 100 μl of the sample is injected into a buffer containing 50 mM PB+100 mM Na₂SO₄ (pH 7.0), and eluted at a flow rate of 0.6 ml/min for 30 minutes. According to the standard curve of HPLC for mRNA quantification, the mRNA in the transcription system is quantified by area integration.

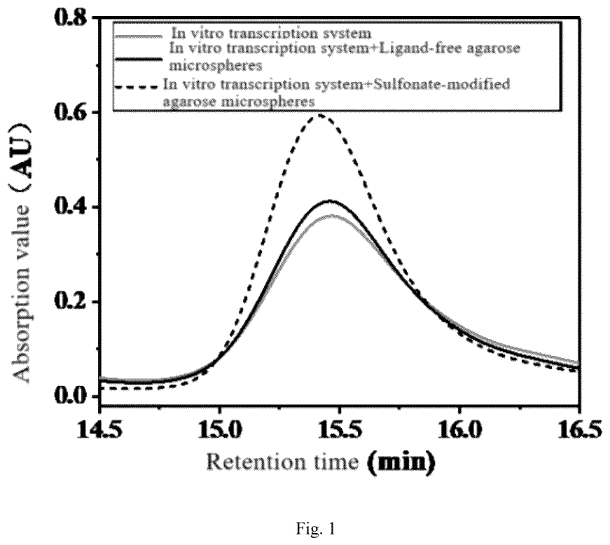

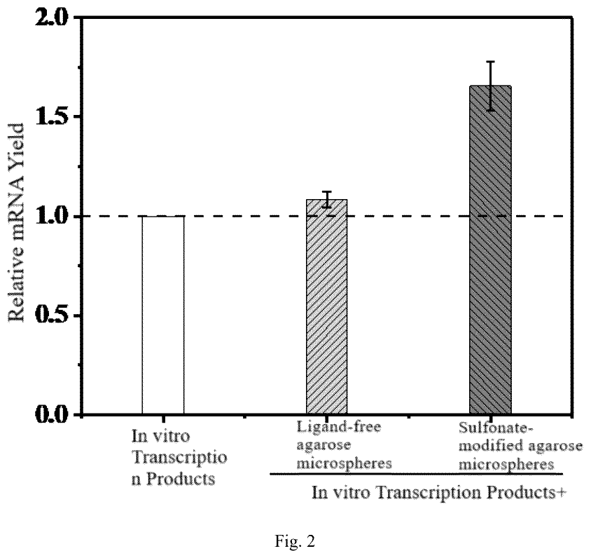

The HPLC chromatograms of Example 3-1, Comparative Example 1 and Comparative Example 2 are shown in FIG. 1, and the relative mRNA yields calculated according to the peak areas are shown in FIG. 2.

The results indicate that adding agarose microsphere solid-phase media modified with sulfonic acid ligands to the transcription system increases the yield of mRNA, while adding agarose microspheres without ligands has no significant impact on the yield of mRNA.

### Example 14 (Test Example 3)

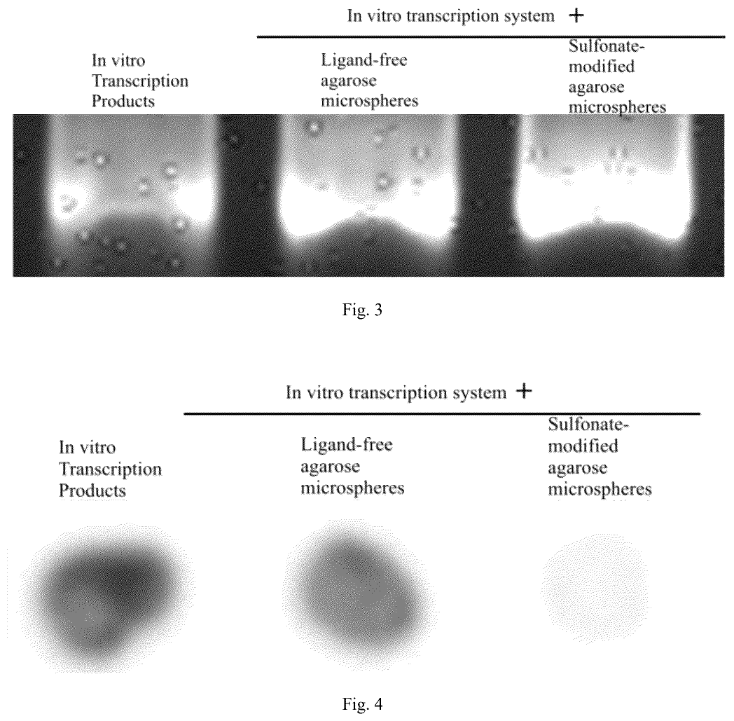

Agarose gel electrophoresis tests are carried out on Example 3-1, Comparative Example 1 and Comparative Example 2. The test method is as follows: Prepare agarose gel with a concentration of 1.5%. Take 3 μl of the transcribed sample, add 7 μl of sterile and enzyme-free water and 2 μl of 6× RNA loading buffer, mix them well. After heating at 65° C. for 5 minutes, immediately load 10 μl of the mixture onto the agarose gel for electrophoresis on ice. The results are shown in FIG. 3.

The results indicate that adding agarose microsphere solid-phase media modified with sulfonic acid ligands to the transcription system increases the yield of mRNA, while adding agarose microspheres without ligands has no significant impact on the yield of mRNA.

### Example 15 (Test Example 4)

The dsRNA yield analysis is performed on Example 3-1, Comparative Example 1 and Comparative Example 2. The test method is as follows: Dot blot method: The transcribed mRNA is diluted to 50 ng/μl. 2 μl of each sample is taken and dropped onto a Nylon membrane. After being air-dried, it is blocked with a blocking solution (5% non-fat dry milk) for 1 hour. The blocking solution is removed and it is washed with TBST buffer three times (10 minutes each time). The primary antibody is added and incubated at room temperature for 1 hour. Then the primary antibody is removed and it is washed with TBST buffer three times (10 minutes each time). The secondary antibody is added and incubated at room temperature for 1 hour. Then the secondary antibody is removed and it is washed with TBST buffer three times (10 minutes each time). TCL luminescence reagents A and B are mixed in a 1:1 ratio. 100 μl is taken and evenly dropped onto the membrane and then pictures are taken through a gel imager. The results are shown in FIG. 4.

The results indicate that adding agarose microsphere solid-phase media modified with sulfonic acid ligands to the in vitro transcription process can significantly reduce the production of dsRNA, while adding agarose microspheres without ligands has no significant impact on the production of dsRNA.

### Example 16

Example 16 is a comparison of the mRNA stability in the transcription systems of Comparative Example 1, Comparative Example 2 and Example 3-1. The difference from Comparative Example 1, Comparative Example 2 and Example 3-1 is that after the transcription is completed, the samples are left at room temperature for 12 hours, and the remaining intact mRNA content is measured and compared with the content right after the transcription is completed.

Table 11 shows the comparison (relative remaining content %) of the mRNA content remaining after being left at room temperature for 12 hours for each sample (Comparative Example 1, Comparative Example 2 and Example 3-1) in Example 16 with the content right after their respective transcriptions are completed. The three samples are represented as “Comparative Example 1-12 h”, “Comparative Example 2-12 h” and “Example Mar. 1, 2012 h” respectively.

TABLE 11

| Process | Relative remaining content of mRNA (%) |
|---|---|
| Comparative example 1-12 h | 76.56% |
| Comparative example 2-12 h | 61.23% |
| Example 3-1-12 h | 98.32% |

The results indicate that after being placed for 12 hours, the mRNA in the in vitro transcription system and the transcription system with agarose microspheres modified without ligands undergoes significant degradation, while the mRNA in the transcription system with agarose microspheres modified with sulfonic acid groups shows almost no degradation. Therefore, adding agarose microspheres modified with sulfonic acid groups to the in vitro transcription system can significantly enhance the stability of mRNA.

### Example 17

### The Medium Balancing Buffer in the Kit is Different:

The method is approximately the same as that described in Example 2, with the difference lying in the different balancing buffers for the solid-phase medium.

Table 12 shows the balancing buffers for the solid-phase medium.

TABLE 12

| | Balancing buffers |
|---|---|
| 1 | 20 mM HEPES buffer, pH 7.4 |
| 2 | 100 mM phosphate buffer, pH 7.0 |
| 3 | 500 mM acetate buffer, pH 6.0 |
| 4 | 200 mM Tris-HCl buffer, pH 7.9 |
| 5 | 300 mM citrate buffer, pH 6.0 |

### Example 18

### The Transcription Reaction Solution in the Kit is Different:

The method is substantially the same as that described in Example 2, with the difference being that the transcription reaction solution is different.

Table 13 shows the compositions of different transcription reaction solutions.

Tabel 13

| | Transcription reaction solutions. |
|---|---|
| 1 | 500 mM Tris-HCl(pH 8.0), 50 mM KCl, |
| | 10 mM MgCl₂, 10 mM DTT |
| 2 | 100 mM HEPES (pH 7.4), 50 mM MgSO₄, 10 mM DTT |
| 3 | 200 mM PB (pH 7.0), 100 mM MgCl₂, 100 mM |
| | NaClO₄, 2 mM mercaptoethanol |
| 4 | 400 mM sodium acetate(pH 6.0), 100 mM MgCl₂, |
| | 100 mM DTT, 20 mM spermidine |

### Comparative Example 4

This Comparative Example 4 is a commercial transcription kit, that is, the kit does not contain solid-phase media.

The operation process is as follows: Take 100 μL of the transcription mixture with the gene of green fluorescent protein as the template: 1) 10 μl of 10× Transcription Buffer (transcription reaction solution): 400 mM Tris-HCl with pH 7.9, 60 mM MgCl₂, 100 mM DTT (dithiothreitol), 20 mM spermidine; 2) 30 μL of green fluorescent protein DNA template; 3) 2 μL each of ATP, CTP, GTP, and UTP; 4) 4 μL of RNase inhibitor (RNA enzyme inhibitor) and 1.5 μL of pyrophosphatase; 5) The amount of T7 RNA Polymerase added is 5 μL; 6) Make up to 100 μL with sterile and enzyme-free water. Gently mix the above components and incubate at 37° C. for 4 hours.

### Test Example 5

The agarose gel electrophoresis method and HPLC method are used to quantify the mRNA in the transcribed samples, and the Dot blot method is used to quantitatively analyze the dsRNA in the in vitro transcription system. The yields of the obtained mRNA and the contents of dsRNA are calculated, and the results are listed in Table 14.

The relative yield of mRNA (%)=the mRNA concentration of the group with solid-phase medium added/the mRNA concentration of the group without solid-phase medium added *100%.

The relative yield of dsRNA (%)=the content of dsRNA in the mRNA of the group with solid-phase medium added/the content of dsRNA in the mRNA of the group without solid-phase medium added *100%.

Table 14 shows the comparison of the statistical results of the relative yield (%) of mRNA and the relative yield (%) of dsRNA obtained in Comparative Example 4 and Example 3-13; among them, the first item in Table 1 of Example 3 is represented as “Example 3-1”, the second item in Table 1 of Example 3 is represented as “Example 3-2”, and so on in sequence.

TABLE 14

| | The relative yield | The relative yield |
|---|---|---|
| Process | of mRNA (%) | of dsRNA (%) |
| Comparative example 4 | 100 | 100 |
| Example 3-1 | 165.79 | 5.12 |
| Example 3-2 | 148.77 | 7.33 |
| Example 3-3 | 150.36 | 7.68 |
| Example 3-4 | 145.98 | 6.87 |
| Example 3-5 | 161.87 | 8.45 |
| Example 4-1 | 143.57 | 10.87 |
| Example 4-2 | 170.12 | 5.02 |
| Example 4-3 | 160.23 | 5.89 |
| Example 4-4 | 152.25 | 7.41 |
| Example 4-5 | 124.82 | 43.25 |
| Example 4-6 | 120.45 | 50.45 |
| Example 5-1 | 135.89 | 12.97 |
| Example 5-2 | 159.72 | 5.22 |
| Example 5-3 | 95.92 | 3.32 |
| Example 5-4 | 85.98 | 1.45 |
| Example 5-5 | 81.63 | 1.23 |
| Example 5-6 | 76.84 | 1.03 |
| Example 6-1 | 165.79 | 5.12 |
| Example 6-2 | 146.72 | 8.41 |
| Example 6-3 | 128.54 | 56.23 |
| Example 6-4 | 110.26 | 72.45 |
| Example 7-1 | 140.89 | 40.73 |
| Example 7-2 | 145.12 | 35.53 |
| Example 7-3 | 148.89 | 35.73 |
| Example 7-4 | 157.89 | 24.73 |
| Example 8-1 | 153.26 | 9.21 |
| Example 8-2 | 142.77 | 9.47 |
| Example 8-3 | 139.26 | 8.91 |
| Example 9-1 | 150.26 | 3.81 |
| Example 9-2 | 162.26 | 2.27 |
| Example 9-3 | 143.26 | 4.91 |
| Example 9-4 | 148.26 | 5.81 |
| Example 10-1 | 159.72 | 5.22 |
| Example 10-2 | 161.72 | 5.72 |
| Example 10-3 | 160.79 | 5.39 |
| Example 11-1 | 138.91 | 5.02 |
| Example 11-2 | 142.91 | 4.85 |
| Example 11-3 | 135.01 | 19.87 |
| Example 17-1 | 138.21 | 6.82 |
| Example 17-2 | 140.36 | 7.91 |
| Example 17-3 | 150.89 | 6.35 |
| Example 17-4 | 145.32 | 7.55 |
| Example 17-5 | 143.11 | 5.31 |
| Example 18-1 | 152.39 | 10.01 |
| Example 18-2 | 141.96 | 15.60 |
| Example 18-3 | 138.22 | 12.98 |
| Example 18-4 | 132.78 | 10.26 |

### Example 19

### This Example is a Comparison Example of the Stability of mRNA in the Transcription Products of Example 3-1 and Comparative Example 4

After the completion of the transcription reactions in Example 3-1 and Comparative Example 4 respectively, the transcription products are placed at room temperature for 12 hours. The intact mRNA in the transcription samples is quantitatively determined by agarose gel electrophoresis and HPLC methods.

Table 15 shows the comparison (relative remaining content %) of the remaining mRNA content in each sample (Example 3-1 and Comparative Example 4) after being placed at room temperature for 12 hours with the content after the completion of their respective transcriptions. The two samples are represented by “Example Mar. 1, 2012 h” and “Comparative Example 4-12 h” respectively.

TABLE 15

| Process | Relative remaining content of mRNA (%) |
|---|---|
| Example 3-1-12 h | 98.32 |
| Comparative Example 4-12 h | 76.56 |

The results show that after being placed for 12 hours, the mRNA in the transcription system of the commercial kit is significantly degraded, while the mRNA in the transcription system with sulfonic acid group-modified agarose microspheres added has almost no degradation. Therefore, the addition of sulfonic acid group-modified agarose microspheres to the kit of the present invention can significantly improve the stability of mRNA.

### Test Example 6

mRNA Quantitative Testing Method: The transcribed samples are subjected to HPLC quantitative analysis using the Arc HPLC series (Waters, USA) on a SEC-2000 (300×7.8 mm) analytical column (Sepax, USA), with the ultraviolet detection wavelength set at 260 nm. In each operation, 100 μl of the sample is injected into a buffer containing 50 mM PB+100 mM Na₂SO₄ (pH 7.0), and eluted at a flow rate of 0.6 ml/min for 30 min. According to the standard curve of HPLC for mRNA quantification, the mRNA in the transcription system is quantified by area integration.

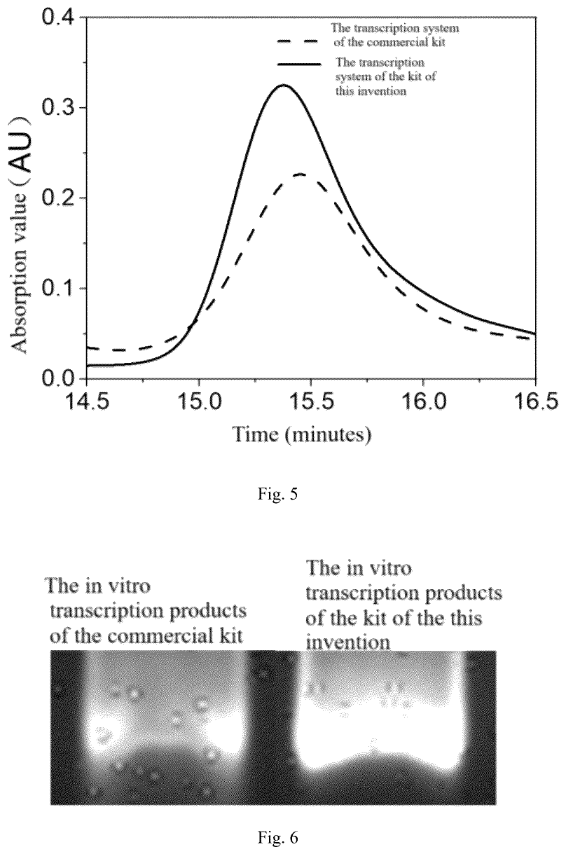

The HPLC chromatograms of Example 3-1 and Comparative Example 4 are shown in FIG. 5.

The results show that the addition of the agarose microsphere solid-phase medium modified with sulfonic acid ligands in the kit of the present invention increases the yield of mRNA.

### Test Example 7

Agarose gel electrophoresis tests are carried out on the samples of the examples and the comparative examples. The test method is as follows: A 1.5% agarose gel is prepared. Then, 3 μl of the transcribed sample is taken and 7 μl of sterile and enzyme-free water as well as 2 μl of 6× RNA loading buffer are added. After mixing them well, the mixture is heated at 65° C. for 5 minutes. Immediately after that, 10 μl of it is loaded onto the agarose gel for electrophoresis on an ice bath. The results are shown in FIG. 6.

The results show that the addition of the agarose microsphere solid-phase medium modified with sulfonic acid ligands in the kit of this invention increases the yield of mRNA.

### Test Example 8

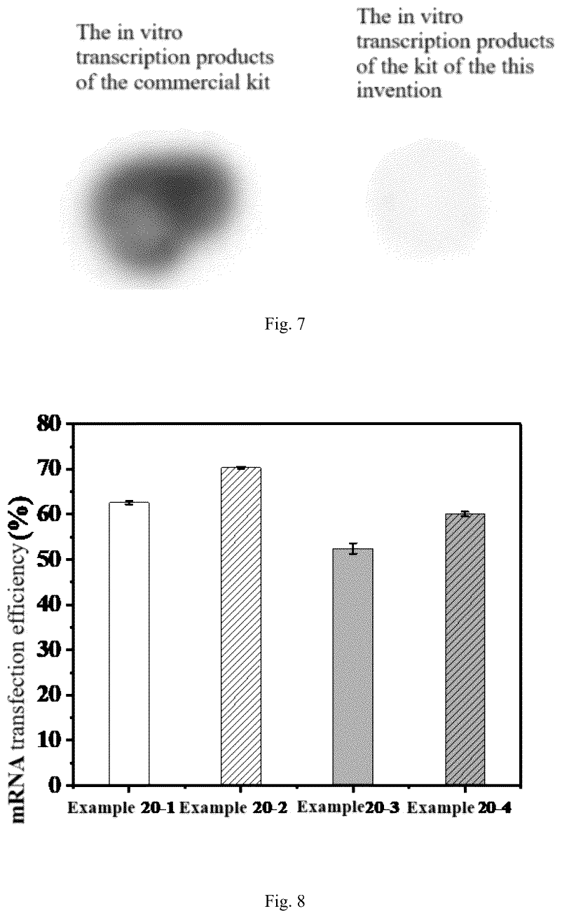

The dsRNA yield of the samples of the examples and the comparative examples is analyzed. The test method is as follows: Dot blot Method: The transcribed mRNA is diluted to 50 ng/μl. 2 μl of each sample is dropped onto a Nylon membrane. After being air-dried, it is blocked with a blocking solution (5% non-fat dry milk) for 1 hour. Then the blocking solution is removed, and the membrane is washed 3 times with TBST buffer (10 minutes each time). The primary antibody is added and incubated at room temperature for 1 hour. After that, the primary antibody is removed, and the membrane is washed 3 times with TBST buffer (10 minutes each time). The secondary antibody is added and incubated at room temperature for 1 hour. Then the secondary antibody is removed, and the membrane is washed 3 times with TBST buffer (10 minutes each time). TCL luminescence reagents A and B are mixed at a ratio of 1:1. 100 μl of the mixture is evenly dropped onto the membrane, and a photo is taken by a gel imager. The results are shown in FIG. 7.

The results show that the addition of the agarose microsphere solid-phase medium modified with sulfonic acid ligands in the kit of this invention can significantly reduce the production of dsRNA.

### Example 20

A method for reducing the formation of dsRNA during the in vitro transcription preparation of mRNA, with the specific steps as follows:

It is substantially the same as the methods described in Comparative Example 1 and Example 9-2. The difference lies in that a cap analogue CAP GAG (3′OMe) is added to the transcription system for co-transcriptional capping to prepare four 100 μL transcription mixtures using the gene of green fluorescent protein as the template.

In the use of the kit, it is roughly the same as that described in Comparative Example 1 and Example 9-2. The difference is that a cap analogue CAP GAG (3′OMe) is added to the transcription system for co-transcriptional capping to prepare four 100 μL transcription mixtures using the gene of green fluorescent protein as the template.

Table 16 shows the transcription systems selected and used in in vitro transcription. The difference between Groups 1-2 and Groups 3-4 in Table 16 lies in whether the modified nucleoside N1-methylpseudouridine (mlw) is used; the difference between Group 1 and Group 2, as well as between Group 3 and Group 4 in Table 16 lies in whether a solid-phase medium is added to the transcription system.

TABLE 16

| Serial | Modification with N1 - | Dextran Microspheres Modified |
|---|---|---|
| Number | Methylpseudouridine (m1ψ) | with Carboxylic Acid Groups |
| 1 | No | No |
| 2 | | Yes (5 mg) |
| 3 | Yes | No |
| 4 | | Yes (5 mg) |

Each reaction system is incubated at 37° C. for 4 hours. After the transcription is completed, the transcription mixtures and microspheres are separated by centrifugation at 12,000 rpm for 2 minutes.

Table 17 shows the comparison of the statistical results of the relative yield (%) of mRNA and the relative yield (%) of dsRNA obtained from the co-transcriptional capping system in this example. Among them, the first item in Table 16 of this example (Example 20) is represented as “Example 20-1”, the second item in Table 16 of this example (Example 20) is represented as “Example 20-2”, and so on for the rest.

TABLE 17

| | Relative Yield | Relative Yield |
|---|---|---|
| Process | of mRNA (%) | of dsRNA (%) |
| Example 20-1 | 100 | 100 |
| Example 20-2 | 105.43 | 7.46 |
| Example 20-3 | 78.17 | 31.34 |
| Example 20-4 | 95.94 | 14.92 |

### Example 21

Cell transfection experiments are carried out on the mRNA obtained in Example 20 after purification through Oligo dT 25 medium. The specific methods are as follows:

Human embryonic kidney 293T cells (HEK293T) are cultured in DMEM medium containing L-glutamine and 10% fetal bovine serum and maintained at 37° C. in a humid incubator with 5% CO₂. The HEK293T cells are seeded in 24-well black plates at a density of 1-3×10⁵/ml and allowed to adhere overnight. Then the capped mRNA is added to the cells: 0.5 μg of mRNA, 1 μl of BOOST, and 1 μl of Trans-IT are successively added to 50 μl of serum-free medium and thoroughly mixed. After the mixture is incubated at room temperature for 2-5 minutes, it is added dropwise to the corresponding wells.

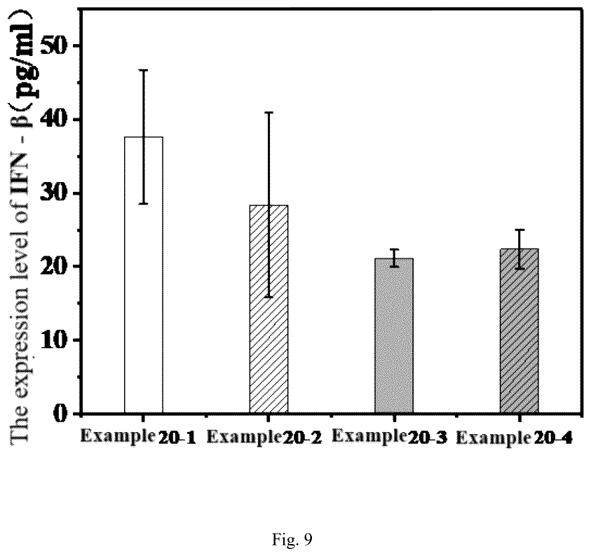

Twenty-four hours after transfection, the expression of EGFP is then determined by flow cytometry (FIG. 8). After transfection, the supernatant of the culture medium is collected and the IFN-β protein level is determined by an ELISA kit (FIG. 9).

The above descriptions are merely preferable examples of this application and are not intended to limit this application. Any modifications, equivalent replacements, improvements, etc. made within the spirit and principles of this application shall all be included within the protection scope of this application.

1. A high-yield preparation method of mRNA for reducing or inhibiting the formation of double-stranded ribonucleic acid during in vitro transcription, wherein a solid-phase medium is added during the transcription process.

2. The high-yield preparation method of mRNA according to claim 1, wherein the said solid-phase medium is a medium modified with negatively charged groups.

3. The high-yield preparation method of mRNA according to claim 2, wherein, the negatively charged groups modified on the solid-phase medium are one or more of sulfonic acid group, methylsulfonic acid group, ethylsulfonic acid group, propylsulfonic acid group, phosphate group, carboxylic acid group, formyl group, hydroxyl group, polyadenylic acid, polythymidylic acid, polyuridylic acid, polyguanylic acid and polycytidylic acid.

4. The high-yield preparation method of mRNA according to claim 1, wherein the form of the solid-phase medium is one or more of granular, membranous and sheet-like.

5. The high-yield preparation method of mRNA according to claim 4, wherein,
granular solid-phase medium includes one or more of microspheres and nanoparticles;
material of the granular solid-phase medium includes one or more of organic material, inorganic material and functional material;
the organic material includes one or more of natural polysaccharides and synthetic polymers;
the natural polysaccharide organic materials include one or more of cellulose, dextran, agarose, chitosan and konjac glucomannan;
the synthetic polymers include one or more of styrenic polymers, acrylic polymers and polyvinyl acid-type polymers;
the inorganic materials include one or more of silica gel, glass, metal oxides and hydroxyapatite;
the functional materials include one or more of magnetic materials and thermos-sensitive materials;
membranous solid-phase medium includes one or more of nitrocellulose membrane, nylon membrane and glass-cellulose membrane;
sheet-like solid-phase medium includes carbon nanotubes.

6. The high-yield preparation method of mRNA according to claim 5, comprising the following steps:
S1. pretreatment of solid-phase medium: washing the solid-phase medium with sterile and nuclease-free water, then equilibrating it with a buffer solution, after shaking for equilibration, draining it by suction; preferably, the buffer solution includes one or more of Tris-HCl buffer, citrate buffer, acetate buffer, phosphate buffer and HEPES buffer; shaking equilibration time is 5-240 minutes;
S2. preparing an in vitro mRNA transcription system and adding the solid-phase medium into the transcription system mixture to carry out transcription reaction for mRNA preparation; the timing of adding the solid-phase medium into the transcription system mixture can be at the transcription initiation stage, during the transcription process, or at the post-transcription stage, preferably at the transcription initiation stage; the amount of the solid-phase medium added is 1-1000 mg/ml, preferably 10-200 mg/ml; the transcription system mixture includes reaction buffer, DNA template, NTP, T7 polymerase, RNase A inhibitor and pyrophosphatase; transcription reaction temperature is 20-60° C.;
S3. after the transcription is completed, collecting the supernatant by centrifugation or gravitational sedimentation methods to obtain the mRNA solution; the centrifugation speed is 8000-12000 rpm/min, preferably 10000 rpm/min; the centrifugation time is 1-3 minutes, preferably 2 minutes.

7. A kit for in vitro transcription synthesis of mRNA capable of reducing or inhibiting the formation of dsRNA, wherein the kit adopts the high-yield preparation method of mRNA for reducing or inhibiting the formation of double-stranded ribonucleic acid during in vitro transcription according to claim 1, the kit comprises a solid-phase medium, a medium equilibration solution/buffer, a transcription reaction solution, a positive control DNA, NTP, T7 polymerase, an RNase A inhibitor, sterile and nuclease-free water, and pyrophosphatase.

8. The kit according to claim 7, wherein, the solid-phase medium is a medium modified with negatively charged groups; the negatively charged groups include one or more of sulfonic acid group, methylsulfonic acid group, ethylsulfonic acid group, propylsulfonic acid group, phosphate group, carboxylic acid group, formyl group, hydroxyl group, polyadenylic acid, polythymidylic acid, polyuridylic acid, polyguanylic acid and polycytidylic acid;
form of the solid-phase medium is one or more of granular, membranous and sheet-like;
granular solid-phase medium includes one or more of microspheres and nanoparticles;
material of the granular solid-phase medium includes one or more of organic materials, inorganic materials and functional materials;
the organic material includes one or more of natural polysaccharides and synthetic polymers;
the natural polysaccharide organic materials include one or more of cellulose, dextran, agarose, chitosan and konjac glucomannan;
the synthetic polymers include one or more of styrenic polymers, acrylic polymers and polyvinyl acid-type polymers;
the inorganic materials include one or more of silica gel, glass, metal oxides and hydroxyapatite;
the functional materials include one or more of magnetic materials and thermos-sensitive materials;
membranous solid-phase medium includes one or more of nitrocellulose membrane, nylon membrane and glass-cellulose membrane;
sheet-like solid-phase medium includes carbon nanotubes;
medium equilibration solution/buffer includes one or more of Tris-HCl buffer, citrate buffer, acetate buffer, phosphate buffer and HEPES buffer;
transcription reaction solution contains a basic buffer, inorganic salts and reducing agents; the basic buffer includes one or more of Tris-HCl buffer, citrate buffer, acetate buffer, phosphate buffer and HEPES buffe; the inorganic salts include one or more of NaCl, KCl, MgCl₂, Na₂SO₄, K₂SO₄, MgSO₄; the reducing agents include one or more of DTT, mercaptoethanol, and reduced glutathione.

9. A method of employing the kit according to claim 8, comprises the following steps:
T1. taking the solid-phase medium from the kit for pretreatment; the pretreatment involves washing the solid-phase medium with sterile and nuclease-free water and then equilibrating it with the medium equilibration solution;
T2. employing the reagents in the kit to prepare an in vitro mRNA transcription system, and adding the pretreated solid-phase medium into the transcription system mixture to carry out the transcription reaction for mRNA preparation;
T3. after the transcription is completed, collecting the supernatant by centrifugation or gravitational sedimentation methods to obtain the mRNA solution.

10. The method of employing the kit according to claim 9, wherein, in step T1, the pretreatment is to wash the solid-phase medium with sterile and nuclease-free water, then equilibrating it with the medium equilibration solution, and draining it after shaking for equilibration; the shaking equilibration time is 5-240 minutes; in step T2, for the pretreated solid-phase medium, the timing of adding it into the transcription system mixture can be at the transcription initiation stage, the transcription process stage, or the post-transcription stage, preferably the transcription initiation stage; the amount of the solid-phase medium added is 1-1000 mg/ml, preferably 10-200 mg/ml; the transcription system mixture includes transcription reaction liquid, DNA template, NTP, T7 polymerase, RNase A inhibitor, and pyrophosphatase; the temperature of the transcription reaction is 20-60° C.
